(19) United States
(12) Patent Application Publication
(10) Pub. No.: US 2021/0401568 A1
(43) Pub. Date: Dec. 30, 2021

(54) LIGAMENT FIXATION DEVICE, LIGAMENT FIXATION SYSTEM, AND LIGAMENT FIXATION METHOD

(71) Applicant: OLYMPUS CORPORATION, Tokyo (JP)

(72) Inventors: Masaki HAYASHI, Tokyo (JP); Toshiya FUJII, Tokyo (JP); Chie ONUMA, Tokyo (JP); Sohei UEDA, Tokyo (JP)

(73) Assignee: OLYMPUS CORPORATION, Tokyo (JP)

(21) Appl. No.: 17/470,764

(22) Filed: Sep. 9, 2021

Related U.S. Application Data
(63) Continuation of application No. PCT/JP2019/009821, filed on Mar. 11, 2019.

(51) Int. Cl. A61F 2/08; A61B 17/32

(52) U.S. Cl. CPC A61F 2/0811; A61B 17/32; A61B 2017/320056; A61F 2002/0852; A61F 2002/0841

(57) ABSTRACT

A ligament fixation method can include forming a bone tunnel to a predetermined depth from a surface of a bone; mounting a reconstruction ligament to the ligament fixation device. The reconstruction ligament can be disposed along outer surfaces of plate-like pressing segments of a ligament fixation device and an outer surface of a connection segment that connects the pressing segments. The method can also include inserting the ligament fixation device into the bone tunnel from the connection-segment side; fastening a screw to a receiving section of the ligament fixation device from an opening of the ligament fixation device to increase a distance between the pair of pressing segments and to compress the reconstruction ligament between each pressing segment and an inner surface of the bone tunnel; and fixing the reconstruction ligament to the bone by causing a part of the screw to dig into the bone.

### CROSS-REFERENCE TO RELATED APPLICATIONS

This is a continuation of International Application PCT/JP2019/009821 which is hereby incorporated by reference herein in its entirety.

### TECHNICAL FIELD

The present disclosure relates to ligament fixation devices, ligament fixation systems, and ligament fixation methods.

### BACKGROUND ART

A known ligament fixation device is used for fixing a reconstruction ligament to a bone tunnel formed in a bone (e.g., see Patent Literature 1).

This fixation device includes a tendon graft guide to which a ligament folded in half is to be hooked and that is to be inserted into a bone tunnel, and also includes a screw to be screwed into a gap between the tendon graft guide and the bone tunnel.

### CITATION LIST

### Patent Literature

### {PTL 1}

U.S. Pat. No. 5,961,520

### SUMMARY OF INVENTION

An aspect of the present disclosure provides a ligament fixation method including forming a bone tunnel in a bone, disposing a reconstruction ligament along an outer surface of a U-shaped ligament fixation device, inserting the ligament fixation device together with the reconstruction ligament into the bone tunnel, fastening a screw from an opening of the ligament fixation device, interposing the reconstruction ligament between the ligament fixation device and the bone by spreading opposing ends of the U-shaped ligament fixation device away from each other, and fixing the reconstruction ligament to the bone by causing a part of the screw to dig into the bone.

Another aspect of the present disclosure provides a ligament fixation device for fixing a reconstruction ligament to a bone tunnel. The ligament fixation device includes a main body having a pair of plate-like pressing segments and a connection segment connecting the pair of opposing pressing segments, and a receiving section that is provided in opposing surfaces of the pair of pressing segments and to which a screw is fastenable in a lengthwise direction of the pressing segments from a distal side of the pressing segments. The receiving section is disposed at a position where the receiving section sets an axis of the screw to be fastened at an intermediate location in a widthwise direction of the connection segment, causes the pair of pressing segments to deform in a direction that increases a distance between the pressing segments when the screw is fastened to the receiving section, and allows a circumferential area of the fastened screw to protrude from at least one side in a widthwise direction of the pressing segments. The reconstruction ligament is fixed to the bone tunnel by inserting the reconstruction ligament disposed along outer surfaces of the pressing segments and the connection segment into the bone tunnel from the connection-segment side and fastening the screw to the receiving section.

### BRIEF DESCRIPTION OF DRAWINGS

FIG. 1 is an exploded perspective view illustrating a ligament fixation system according to an embodiment of the present disclosure.

FIG. 2 is a perspective view illustrating the ligament fixation system in FIG. 1 in an assembled state.

FIG. 3 is a perspective view illustrating a ligament fixation device and a screw of the ligament fixation system in FIG. 1.

FIG. 4 is a vertical sectional view illustrating a state before a reconstruction ligament is attached to the ligament fixation device in FIG. 2 and inserted into a bone tunnel.

FIG. 5 is a vertical sectional view illustrating a state where the reconstruction ligament and the ligament fixation device are inserted into the bone tunnel from the state in FIG. 4.

FIG. 6 is a vertical sectional view illustrating a state where the screw is fastened between pressing segments of the ligament fixation device from the state in FIG. 5.

FIG. 7 is a vertical sectional view illustrating a state where an installation jig has been removed from the state in FIG. 6.

FIG. 8 is a cross-sectional view illustrating the state in FIG. 6.

FIG. 9 is a cross-sectional view illustrating a modification of FIG. 8.

FIG. 10 is a flowchart illustrating a ligament fixation method according to an embodiment of the present disclosure.

FIG. 11 is a partial perspective view illustrating an anterior cruciate ligament originally existing in a knee joint.

FIG. 12 is a vertical sectional view illustrating the location and angle of a thigh bone tunnel formed in a thigh bone.

### DESCRIPTION OF EMBODIMENTS

A ligament fixation device 1, a ligament fixation system 100, and a ligament fixation method according to an embodiment of the present disclosure will be described below with reference to the drawings.

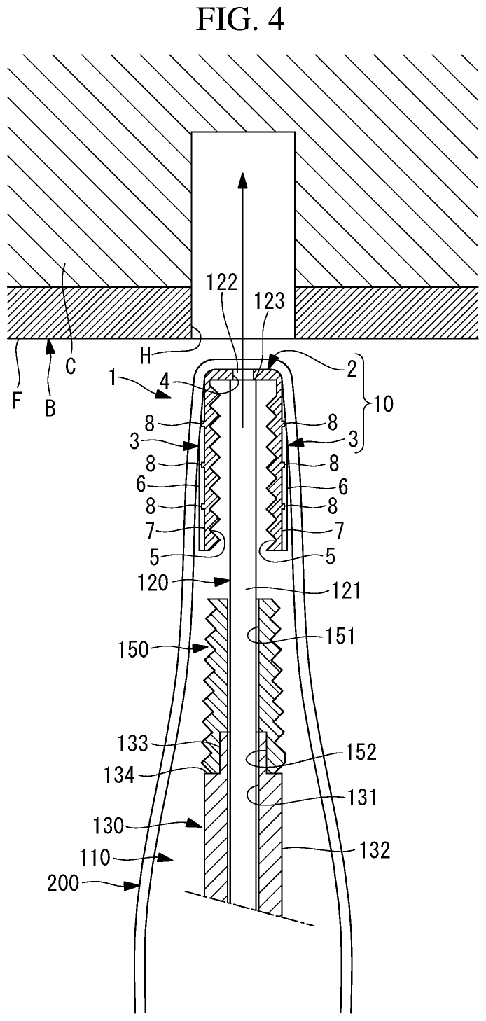

When mending the anterior cruciate ligament or the posterior cruciate ligament of a knee joint damaged as a result of excessive stress applied thereto during sports activity, for example, a tendon autograft is extracted as a ligament graft and is used as a reconstruction ligament (see FIG. 4). Alternatively, an artificial ligament that has been artificially fabricated may be used. Normally, a reconstruction ligament 200 to be used is obtained by connecting artificial ligaments or high-strength sutures as tension threads to the opposite ends of a ligament graft.

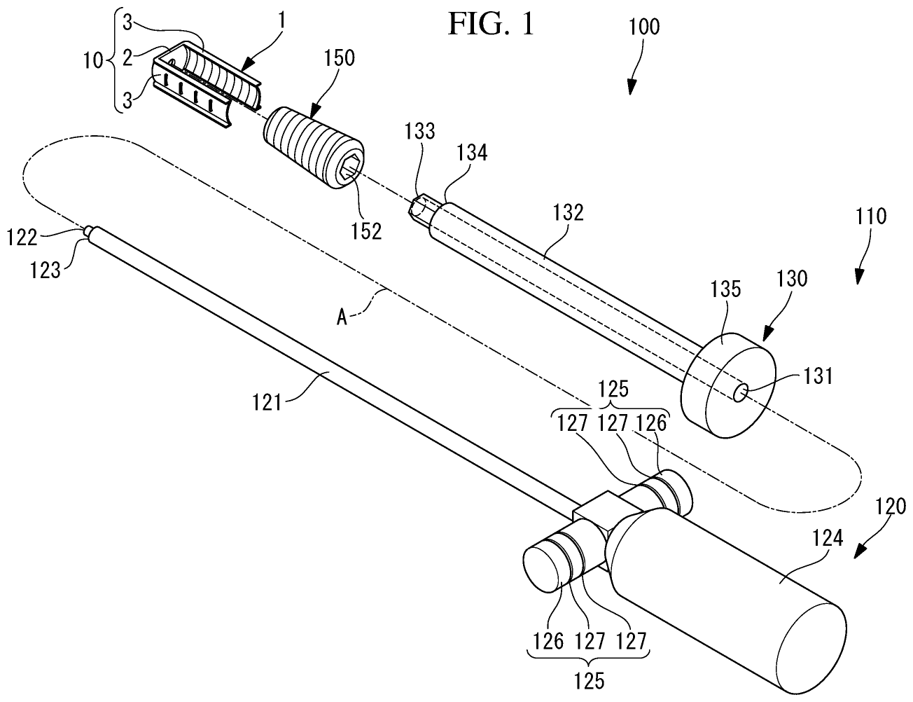

As shown in FIG. 1, the ligament fixation system 100 according to this embodiment includes the ligament fixation device 1, a screw 150, and an installation jig 110.

The ligament fixation device 1 and the screw 150 according to this embodiment are composed of a material having high biocompatibility and sufficient strength.

The biocompatible material used in the ligament fixation device 1 and the screw 150 is preferably a metallic material that can ensure sufficient strength and resiliency over a long period of time, and may be freely selected from, for example, stainless steel (such as SUS316L), pure titanium, and a titanium alloy. Alternatively, the biocompatible material used in the ligament fixation device 1 and the screw 150 may be a bioabsorbable material, such as polylactate (PLLA), polyglycolide (PGA), or a magnesium alloy, or may be a highly functional resin, such as polyether ether ketone.

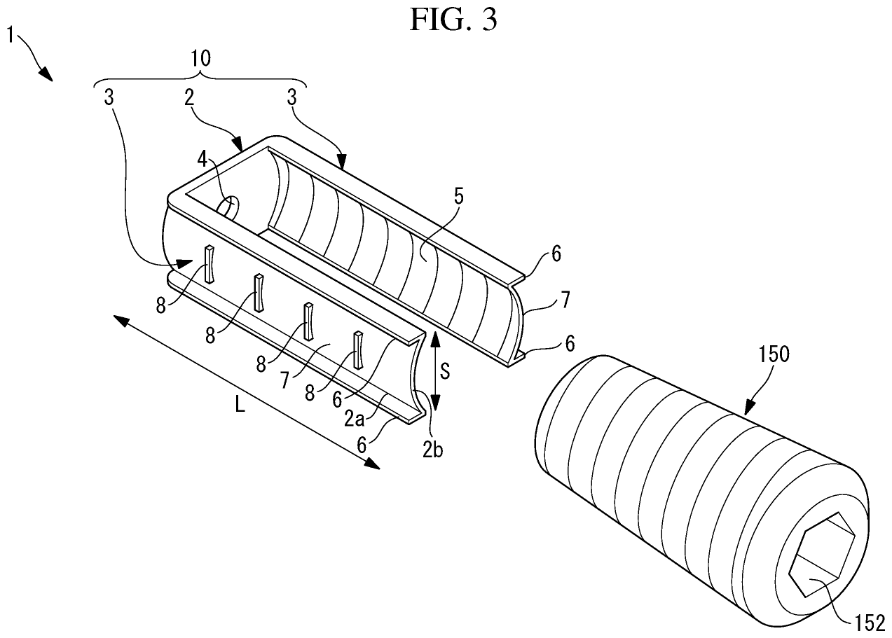

As shown in FIG. 3, the ligament fixation device 1 is U-shaped and has a main body 10 constituted of a rectangular plate-like connection segment 2 having long (lengthwise) sides 2a and short (widthwise) sides 2b and a pair of plate-like pressing segments 3 extending in a cantilevered manner from the opposite lengthwise ends of the connection segment 2 in a direction orthogonal to the connection segment 2. Specifically, the connection segment 2 is a member connected with the proximal ends of the pair of pressing segments 3. The U-shape of the ligament fixation device 1 may be such that connection areas between the connection segment 2 and the pressing segments 3 are circular-arc-shaped or are orthogonal to each other. Furthermore, the connection segment 2 and the pressing segments 3 may be integrated with each other, or the connection segment 2 and the pressing segments 3 may be separate components and fastened to each other with screws.

The connection segment 2 has a cross-sectional shape that is slightly smaller than the cross section of a bone tunnel H to be formed in a bone B, to be described later. The center of the connection segment 2 is provided with a through-hole 4 that extends therethrough in the thickness direction and that is to be engaged with a columnar section 122 at the distal end of a first shaft 121 of the installation jig 110.

The opposing inner surfaces of the pair of pressing segments 3 are provided with a receiving section (i.e., female threaded section) 5 for fastening the screw 150 thereto. The outer surfaces of the pressing segments 3 are each provided with a pair of sidewalls 6 extending parallel to each other in a lengthwise direction L along opposite edges in a widthwise direction S. The outer surface and the pair of sidewalls 6 of each pressing segment 3 form a recess 7 for partially accommodating the reconstruction ligament 200 therein. The receiving section 5 is not limited to having a screw-like structure, and may be a claw-like fastener that prevents the installation jig 110 from falling off.

The outer surface of each pressing segment 3 that constitutes the recess 7 of the pressing segment 3 is provided with a plurality of protrusions 8 protruding outward and arranged at a distance from each other in the lengthwise direction.

The axis of the receiving section 5 is disposed at the center of the connection segment 2 in the widthwise direction and extends parallel to the pressing segments 3.

As shown in FIG. 3, the screw 150 is a tapered screw that tapers toward the distal end thereof. As shown in FIG. 4, the screw 150 is provided with a center hole 151 extending therethrough in the longitudinal direction along the central axis. One end (i.e., rear end) of the center hole 151 is provided with a hexagonal hole (i.e., torque applier) 152 extending to an intermediate location of the center hole 151 in the depth direction.

The screw 150 has an outer diameter that is sufficiently larger than the width of the connection segment 2 and the pressing segments 3. Accordingly, when the screw 150 is fastened from the distal side of the pressing segments 3 to the receiving section 5 provided at the inner surfaces of the pair of pressing segments 3, the pressing segments 3 (i.e., the ends of the U-shaped ligament fixation device 1) are elastically deformed and are spread away from each other, and circumferential areas of the screw 150 are caused to protrude from the opposite widthwise sides of the pressing segments 3.

As shown in FIG. 1, the installation jig 110 includes a first jig 120 and a second jig 130.

The first jig 120 includes the first shaft 121, which is rod-like, the columnar section 122 and a step 123 provided at one end of the first shaft 121, and a handle 124 provided at the other end of the first shaft 121.

The first shaft 121 has an outer diameter that is smaller than the width of the connection segment 2 of the ligament fixation device 1 and the inner diameter of the center hole 151 of the screw 150.

The columnar section 122 has a cylindrical shape that engages with the through-hole 4 provided in the connection segment 2 of the ligament fixation device 1. The outer diameter of the columnar section 122 is smaller than the outer diameter of the first shaft 121, and the step 123 is disposed where the step 123 abuts on the inner surface of the connection segment 2 when the columnar section 122 is engaged with the through-hole 4.

The handle 124 has a cylindrical shape with an outer diameter sufficiently larger than the first shaft 121.

As shown in FIG. 1, the handle 124 is provided with a thread securing section 125 near the first shaft 121. The thread securing section 125 is where the tension threads at the opposite ends of the reconstruction ligament 200 mounted to the ligament fixation device 1 are to be secured. The thread securing section 125 is provided with a plurality of circumferential grooves 127 in two columnar sections 126 extending orthogonally to a longitudinal axis A of the first shaft 121. The circumferential grooves 127 are provided for winding and securing the tension threads thereto.

The second jig 130 includes a cylindrical second shaft 132 having an inner hole 131 through which the first shaft 121 is extendable in a movable manner along the longitudinal axis A and in a rotatable manner around the longitudinal axis A. The second jig 130 also includes a hexagonal columnar section (i.e., connector) 133 and a step 134 that are provided at one end of the second shaft 132 and that are detachably engageable with the hexagonal hole 152 in the screw 150, and a handle 135 that is provided at the other end of the second shaft 132 and that supplies the second shaft 132 with torque around the longitudinal axis A. The handle 135 may have a shape easily grippable by a user and is desirably subjected to an embossing treatment or a non-slip treatment.

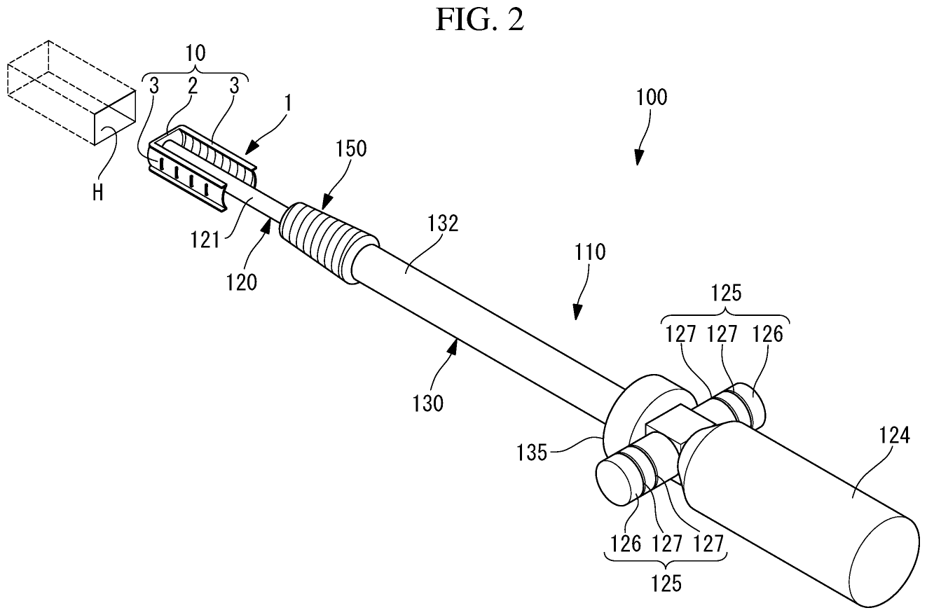

As shown in FIG. 2, the first shaft 121 is inserted into the inner hole 131 of the second shaft 132 from the proximal end in a state where the hexagonal columnar section 133 having a cross-sectional shape complementary to that of the hexagonal hole 152 is engaged with the hexagonal hole 152 and the step 134 is in abutment with the rear end of the screw 150. The first shaft 121 has enough length to extend through the center hole 151 of the screw 150 and support the screw 150 in a rotatable manner around the longitudinal axis A, and also to protrude from the distal end of the screw 150 and allow the columnar section 122 to engage with the through-hole 4 of the ligament fixation device 1 forward of the distal end of the screw 150.

The ligament fixation method according to this embodiment using the ligament fixation device 1 and the ligament fixation system 100 having the above-described configuration will be described below.

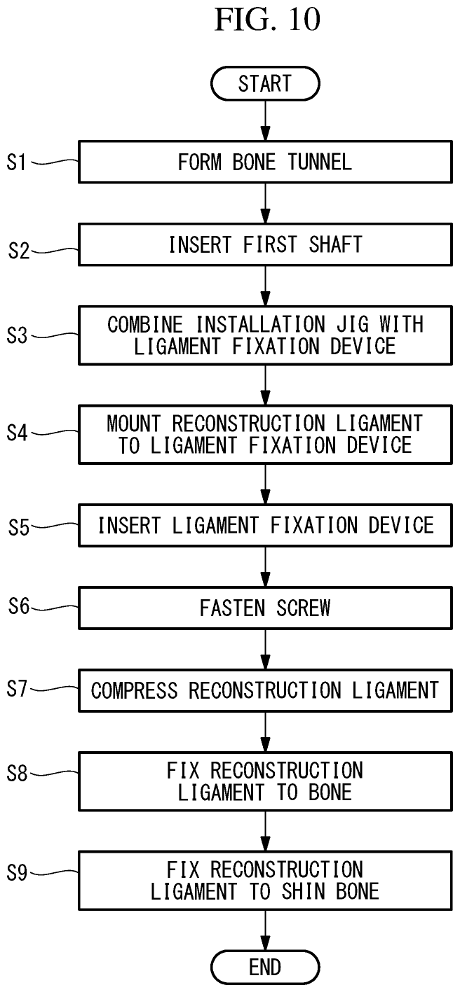

As shown in FIG. 10, the ligament fixation method according to this embodiment is for fixing the reconstruction ligament 200 to the bone B.

First, a bone tunnel H having a predetermined depth is formed in the bone (such as a thigh bone) B (step S1). As shown in FIG. 4, the bone tunnel H in the bone B is formed to extend through cortical bone F to a depth where cancellous bone C exists.

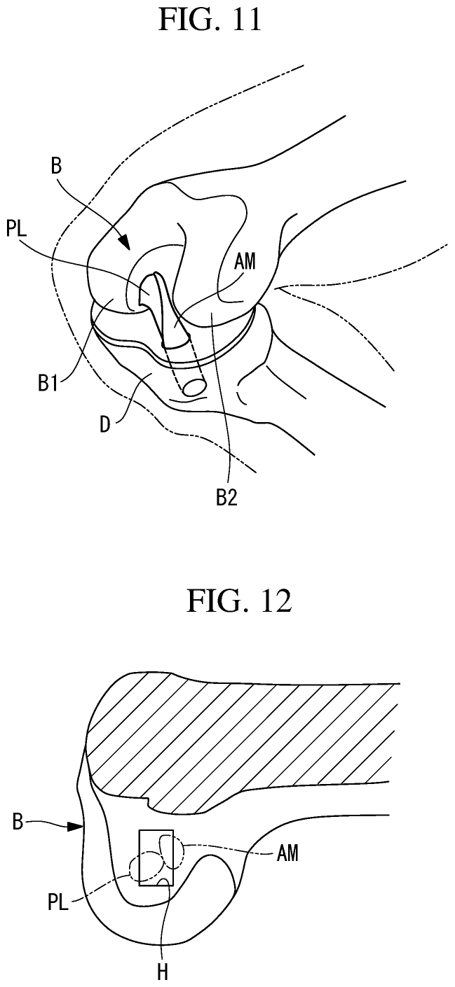

As shown in FIG. 11, an anterior cruciate ligament originally connects an inner location of a lateral condyle B1 disposed at the shin-bone-D end of the thigh bone B with a substantially central location on the inner front side of an inner joint surface of the shin bone D that receives a medial condyle B2 disposed next to the inner side of the lateral condyle B1.

Anatomically, the anterior cruciate ligament is further divided into an antero-medial bundle AM and a postero-medial bundle PL. As indicated by chain lines in FIG. 12, the antero-medial bundle AM and the postero-medial bundle PL are attached substantially next to each other in the longitudinal direction of the thigh bone B at the inner location of the lateral condyle B1.

As indicated by a solid line in FIG. 12, after the damaged anterior cruciate ligament has been removed, the thigh bone tunnel H is formed at the inner location of the lateral condyle B1 originally connected with the anterior cruciate ligament, so as to extend from the surface of the lateral condyle B1 to a depth where the cancellous bone C exists inside. Because the orientation of the bone tunnel H is to be freely set by the user, the orientation of the bone tunnel H is not limited to that shown in FIG. 12.

The bone tunnel H has a fixed rectangular (i.e., noncircular) cross-sectional shape that is slightly larger than the outer rectangular shape of the connection segment 2 of the ligament fixation device 1. Specifically, the bone tunnel H has a maximum inner dimension that is slightly larger than the length of the connection segment 2, and has a minimum inner dimension that is slightly larger than the width of the connection segment 2. Accordingly, the ligament fixation device 1 can be easily inserted into the bone tunnel H in a state where the thickness direction of the connection segment 2 is aligned with the axis of the bone tunnel H.

The bone tunnel H having the rectangular cross-sectional shape can be easily formed by using, for example, an ultrasonic probe described in WO 2018/078826. As indicated by a dashed line in FIG. 11, by using a known technique, a bone tunnel extending toward the shin bone D is formed in the shin bone D at the substantially central location on the inner front side of the inner joint surface originally connected with the anterior cruciate ligament.

Subsequently, as shown in FIG. 2, the hexagonal columnar section 133 at the distal end of the second shaft 132 of the second jig 130 is engaged with the hexagonal hole 152 of the screw 150, and the first shaft 121 of the first jig 120 is inserted into the inner hole 131 of the second shaft 132 and the center hole 151 of the screw 150 (step S2). Then, the columnar section 122 of the first jig 120 is engaged with the through-hole 4, so that the installation jig 110 and the ligament fixation device 1 are combined with each other in a state where the inner surface of the connection segment 2 is in abutment with the step 123 of the first jig 120 (step S3).

With respect to the ligament fixation device 1 assembled in this manner, the reconstruction ligament 200 is bent in half and is looped around the outer surfaces of the pair of pressing segments 3 and the connection segment 2, as shown in FIG. 4. Then, while fixed tension is being applied to the reconstruction ligament 200, the tension threads at the opposite ends thereof are wound around the thread securing section 125 of the first jig 120. Consequently, the reconstruction ligament 200 is mounted to the ligament fixation device 1 (step S4).

Because the pressing segments 3 are provided with the recesses 7 for partially accommodating the reconstruction ligament 200 therein, the reconstruction ligament 200 is prevented from being detached from the pressing segments 3 in the widthwise direction, so as to be maintained in a mounted state to the ligament fixation device 1. Moreover, the ligament fixation device 1 is attached to the distal end of the first shaft 121 while being prevented from falling off by means of the tensioned reconstruction ligament 200.

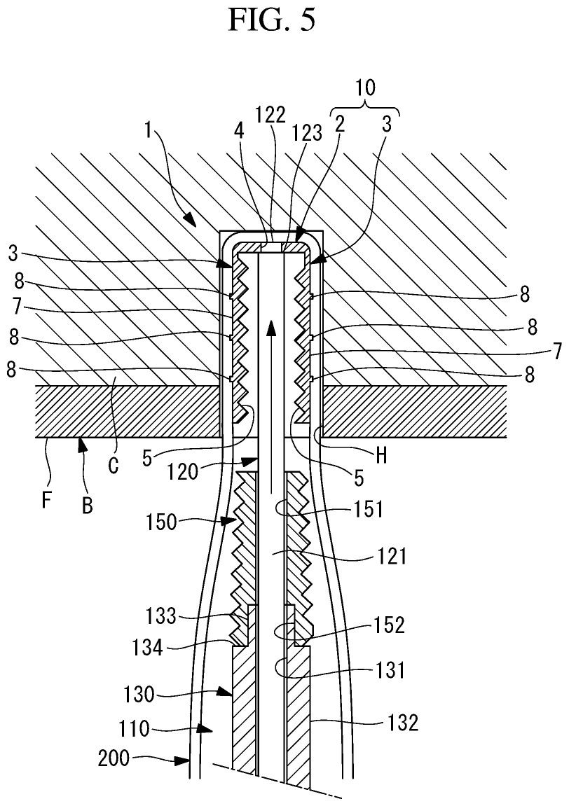

As shown in FIG. 5, the ligament fixation device 1 equipped with the reconstruction ligament 200 attached to the distal end of the first shaft 121 of the installation jig 110 in this manner is inserted into the bone tunnel H (step S5).

In this case, the ligament fixation device 1 is inserted into the bone tunnel H from the connection segment 2 side (i.e., the closed end of the ligament fixation device 1) in a state where the thickness direction of the connection segment 2 is aligned with the axial direction of the bone tunnel H.

When the ligament fixation device 1 is sufficiently inserted into the bone tunnel H, as shown in FIG. 5, the second jig 130 is moved forward along the longitudinal axis A relative to the first jig 120, and the handle 135 of the second jig 130 is supplied with torque around the longitudinal axis A. Accordingly, the screw 150 attached to the distal end of the second jig 130 rotates around the longitudinal axis A while being moved forward using the first shaft 121 as a guide, whereby the screw 150 is fastened to the receiving section 5 at the inner surfaces of the pressing segments 3 from the distal side of the pressing segments 3 of the ligament fixation device 1 (i.e., the open end of the ligament fixation device) disposed within the bone tunnel H (step S6).

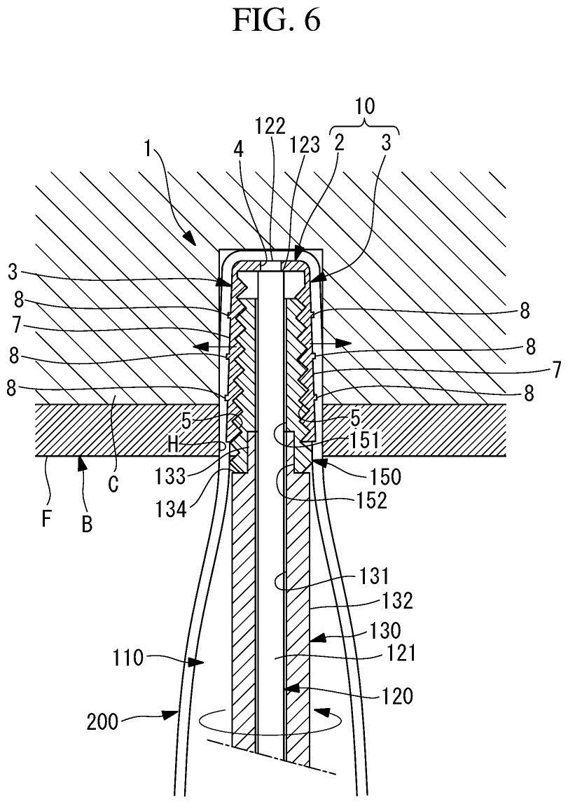

Because the screw 150 is a tapered screw, the pair of opposing pressing segments 3 shift in a direction in which the distance therebetween (i.e., the distance between the opposing ends of the U-shaped ligament fixation device 1) increases as the screw 150 becomes fastened to the receiving section 5, as shown in FIG. 6. Accordingly, the distance between the outer surface of each pressing segment 3 and the inner surface of the bone tunnel H decreases, so that the reconstruction ligament 200 looped around the outer surfaces of the pressing segments 3 is compressed between the pressing segments 3 and the bone B (step S7).

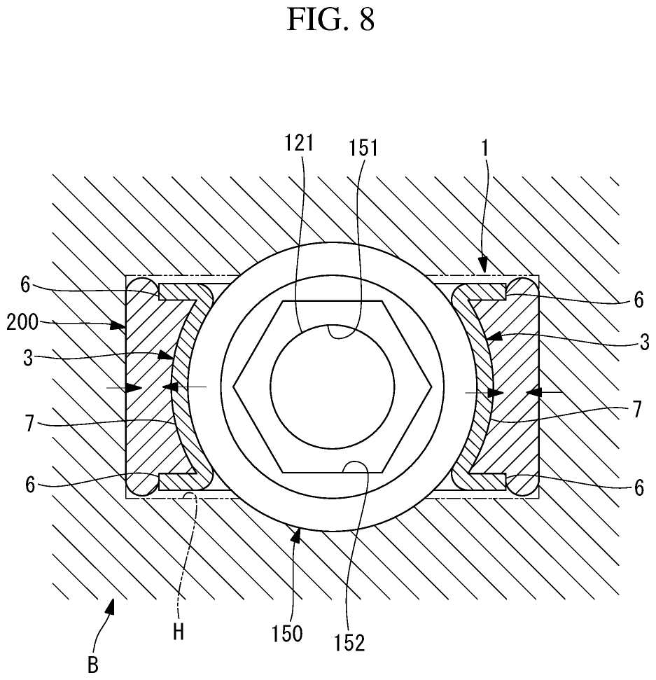

Furthermore, in this embodiment, the outer diameter of the screw 150 is larger than the width of each pressing segment 3, and the circumferential areas of the screw 150 protrude from the opposite widthwise sides of the pressing segments 3 in a state where the screw 150 is fastened to the receiving section 5. Accordingly, the screw 150 protruding from the pressing segments 3 partially digs into the inner surface of the bone tunnel H, as shown in FIG. 8.

As a result, the ligament fixation device 1 is fixed within the bone tunnel H more reliably in accordance with the engagement between the inner surface of the bone tunnel H and the screw 150. Furthermore, the reconstruction ligament 200 is compressed between each pressing segment 3 of the ligament fixation device 1 fixed within the bone tunnel H in this manner and the bone tunnel H, so that friction therebetween is increased. In particular, the protrusions 8 provided on the outer surfaces of the pressing segments 3 dig into the reconstruction ligament 200, so that a greater fixation force is produced, whereby the reconstructed ligament 200 can be prevented from falling out of the bone tunnel H.

In this case, in the ligament fixation device 1 and the ligament fixation system 100 according to this embodiment, the longitudinal axis of the screw 150 is disposed at the center of the connection segment 2 (i.e., an intermediate location in the widthwise direction and the lengthwise direction). This is advantageous in that, even if the reconstructed ligament 200 repeatedly receives tension acting in the axial direction of the bone tunnel H, a moment of force around an axis orthogonal to the axis of the bone tunnel H is less likely to act on the ligament fixation device 1, so that the ligament 200 can be supported in a well-balanced manner and the fixed ligament 200 can be prevented from loosening.

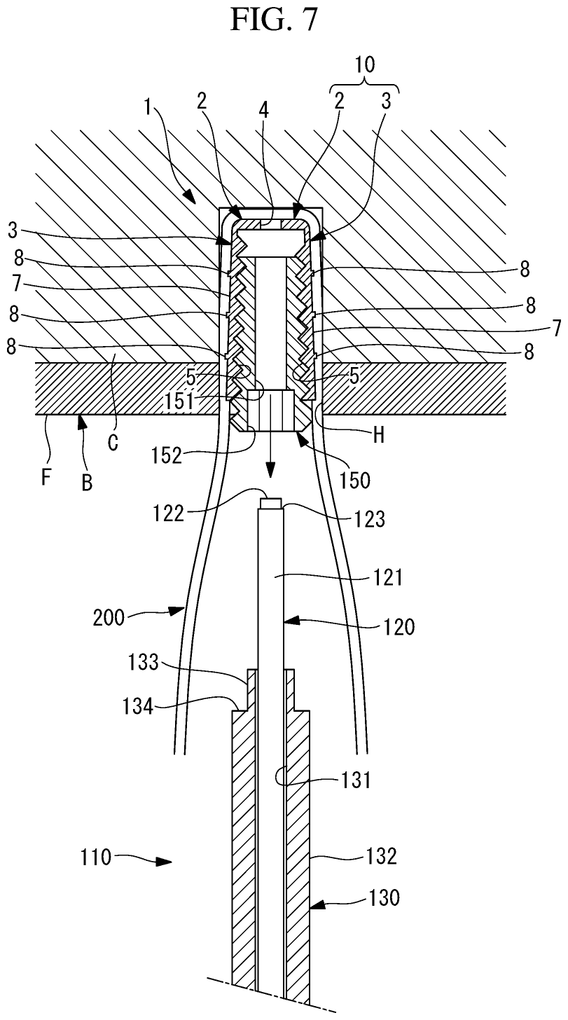

Subsequently, the tension threads are removed, and the installation jig 110 is removed from the bone tunnel H, as shown in FIG. 7, so that the ligament fixation device 1 equipped with the reconstruction ligament 200 is retained inside the bone tunnel H, and the reconstruction ligament 200 is fixed to the bone B (step S8). Alternatively, in order to facilitate the operation of the handle 135 involved in the fastening of the screw 150, the tension threads may be removed between step S5 and step S6.

The two pieces of the reconstruction ligament 200 fixed to the thigh bone B are inserted into the bone tunnel in the shin bone D and are fixed to the shin bone D in a tensioned state (step S9). The two pieces of the reconstruction ligament 200 are fixed to the shin bone D by using, for example, a known technique described in Japanese Unexamined Patent Application, Publication No. 2018-117905. Consequently, the anterior cruciate ligament is reconstructed.

Although the bone tunnel H and the connection segment 2 are described as being rectangular in this embodiment, the configuration is not limited to this.

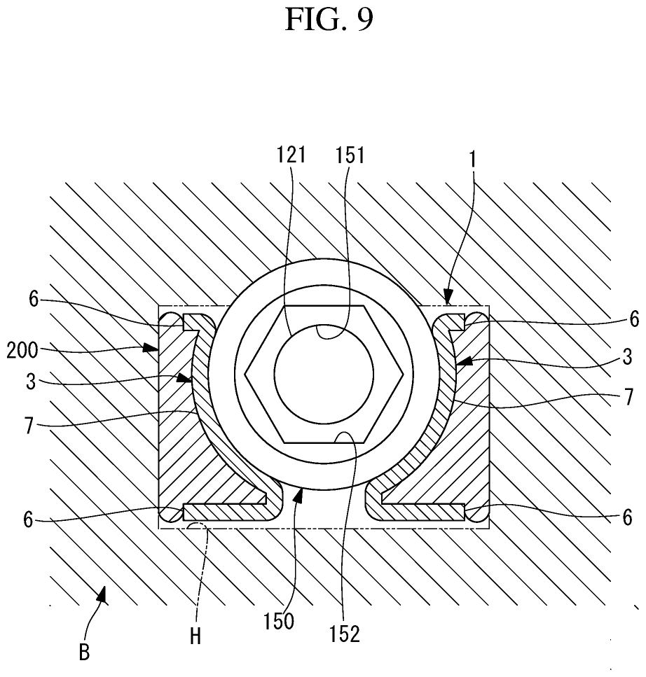

Moreover, although the axis of the screw 150 is aligned with the center of the connection segment 2, the axis of the screw 150 may be offset toward one side in the widthwise direction of the connection segment 2, as shown in FIG. 9.

A sufficient fixation force can be obtained even in a case where the screw 150 digs into the inner surface of the bone tunnel H toward only one side in the widthwise direction of the pressing segments 3. Moreover, even if the axis of the screw 150 is not disposed at the center, the occurrence of a moment of force can be reduced and the reconstruction ligament 200 can be supported in a well-balanced manner so long as the axis of the screw 150 is disposed at an intermediate location of the connection segment 2 in the widthwise direction.

Furthermore, the connection segment 2 or the pressing segments 3 of the ligament fixation device 1 may be designed for temporarily fixing the reconstruction ligament 200. For example, the connection segment 2 or the pressing segments 3 may be provided with a suture through-hole for tying the reconstruction ligament 200 thereto by using a suture, or may be provided with a hole that allows the reconstruction ligament 200 to extend therethrough.

The shape and the number of protrusions 8 provided on the outer surfaces of the pressing segments 3 may be freely determined.

Moreover, if it is difficult to supply the handle 135 of the second jig 130 with torque in a state where the tension threads remain fixed to the thread securing section 125, the tension threads may be removed from the thread securing section 125 when the screw 150 starts to engage with the receiving section 5, and the handle 135 may be subsequently supplied with torque.

The above-described embodiment leads to the following aspects.

An aspect of the present disclosure provides a ligament fixation method including forming a bone tunnel in a bone, disposing a reconstruction ligament along an outer surface of a U-shaped ligament fixation device, inserting the ligament fixation device together with the reconstruction ligament into the bone tunnel, fastening a screw from an opening of the ligament fixation device, interposing the reconstruction ligament between the ligament fixation device and the bone by spreading opposing ends of the U-shaped ligament fixation device away from each other, and fixing the reconstruction ligament to the bone by causing a part of the screw to dig into the bone.

According to this aspect, the reconstruction ligament is disposed in the formed bone tunnel along the outer surface of the U-shaped ligament fixation device, and the ligament fixation device and the reconstruction ligament are inserted together into the bone tunnel. Accordingly, the reconstruction ligament is disposed between the ligament fixation device and the inner surface of the bone tunnel.

In this state, the screw is fastened from the opening of the ligament fixation device. Because the ligament fixation device is U-shaped, when the screw is fastened thereto, the opposing ends of the U-shape are spread away from each other by the screw. Accordingly, the reconstruction ligament is compressed and interposed between the ligament fixation device and the inner surface of the bone tunnel. Then, a part of the fastened screw digs into the inner surface of the bone tunnel.

In this case, in this aspect, even if the reconstruction ligament disposed along the outer surface of the ligament fixation device repeatedly receives tension acting in the axial direction of the bone tunnel after the ligament is reconstructed, a moment of force around an axis orthogonal to the axis of the screw is less likely to act on the ligament fixation device. As a result, the reconstruction ligament can be supported in a well-balanced manner.

Specifically, with the ligament fixation device according to this aspect, the compression of the reconstruction ligament between the ligament fixation device and the inner surface of the bone tunnel and the fixation of the ligament fixation device to the bone tunnel can be realized at the same time by fastening the screw, so that the reconstruction ligament can be fixed to the bone tunnel more reliably.

In the above aspect, the ligament fixation method may further include: forming the bone tunnel to a predetermined depth from a surface of the bone; mounting the reconstruction ligament, disposed along outer surfaces of a pair of plate-like pressing segments of the ligament fixation device and an outer surface of a connection segment connecting the pair of opposing pressing segments, to the ligament fixation device and inserting the ligament fixation device into the bone tunnel from the connection-segment side; and fastening the screw to a receiving section of the ligament fixation device to increase a distance between the pair of pressing segments and to compress the reconstruction ligament between each pressing segment and an inner surface of the bone tunnel, and causing the screw protruding in a widthwise direction of the pressing segments to dig into the inner surface of the bone tunnel.

Furthermore, in the above aspect, the bone tunnel may have a rectangular cross-sectional shape that is slightly larger than an outer shape of the connection segment.

Another aspect of the present disclosure provides a ligament fixation device for fixing a reconstruction ligament to a bone tunnel. The ligament fixation device includes a main body having a pair of plate-like pressing segments and a connection segment connecting the pair of opposing pressing segments, and a receiving section that is provided in opposing surfaces of the pair of pressing segments and to which a screw is fastenable in a lengthwise direction of the pressing segments from a distal side of the pressing segments. The receiving section is disposed at a position where the receiving section sets an axis of the screw to be fastened at an intermediate location in a widthwise direction of the connection segment, causes the pair of pressing segments to deform in a direction that increases a distance between the pressing segments when the screw is fastened to the receiving section, and allows a circumferential area of the fastened screw to protrude from at least one side in a widthwise direction of the pressing segments. The reconstruction ligament is fixed to the bone tunnel by inserting the reconstruction ligament disposed along outer surfaces of the pressing segments and the connection segment into the bone tunnel from the connection-segment side and fastening the screw to the receiving section.

According to this aspect, the ligament fixation device having the reconstruction ligament disposed along the outer surfaces of the connection segment and the pressing segments is inserted from the connection-segment side into the bone tunnel formed to the predetermined depth from the surface of the bone at a position where the reconstruction ligament is to be fixed. Accordingly, the reconstruction ligament is disposed between the inner surface of the bone tunnel and the pair of opposing pressing segments, as well as between the inner surface of the bone tunnel and the connection segment.

In this state, the screw is inserted in the axial direction of the bone tunnel into between the pair of pressing segments whose distal ends are disposed at the opening of the bone tunnel, and is fastened to the receiving section secured to the inner surfaces of the pressing segments. Because the pressing segments are connected to the connector in a cantilevered fashion, the distal ends of the pressing segments are shifted away from each other by the screw when the screw is fastened. Accordingly, the reconstruction ligament is compressed between each pressing segment and the inner surface of the bone tunnel.

Furthermore, the circumferential area of the screw fastened to the receiving section is disposed where the circumferential area protrudes from at least one side in the widthwise direction of the pressing segments. Therefore, by forming a bone tunnel with a cross-sectional shape to which the ligament fixation device equipped with the reconstruction ligament fits tightly, the circumferential area of the screw protruding from the pressing segments can dig into the inner surface of the bone tunnel.

In this case, according to this aspect, the axis of the screw is disposed at an intermediate location of the connection segment in the widthwise direction. Therefore, even if the reconstruction ligament looped around the outer surface of the connection segment repeatedly receives tension acting in the axial direction of the bone tunnel after the ligament is reconstructed, a moment of force around an axis orthogonal to the axis of the screw is less likely to act on the ligament fixation device. As a result, the reconstruction ligament can be supported in a well-balanced manner.

Specifically, with the ligament fixation device according to this aspect, the compression of the reconstruction ligament between each pressing segment and the inner surface of the bone tunnel and the fixation of the ligament fixation device to the bone tunnel can be realized at the same time by fastening the screw, so that the reconstruction ligament can be fixed to the bone tunnel more reliably.

In the above aspect, the receiving section may be disposed at a position where the receiving section sets the axis of the screw to be fastened at a central location in the widthwise direction of the connection segment and allows the fastened screw to protrude from opposite sides in the widthwise direction of the pressing segments.

According to this configuration, the screw can dig into the inner surface of the bone tunnel at the opposite widthwise sides of the pressing segments, so that the reconstruction ligament can be fixed to the bone tunnel more reliably in a well-balanced manner.

Furthermore, in the above aspect, the outer surface of each pressing segment may be provided with a recess that partially accommodates the reconstruction ligament in the lengthwise direction of the pressing segment.

According to this configuration, the reconstruction ligament looped around the outer surfaces of the pressing segments is partially accommodated within the recesses, so that the reconstruction ligament can be prevented from falling out of the pressing segments in the widthwise direction in the process of being fixed within the bone tunnel.

Another aspect of the present disclosure provides a ligament fixation system including the aforementioned ligament fixation device and the screw.

In the above aspect, the screw may have a diameter that is larger than a widthwise dimension of the pair of pressing segments.

According to this configuration, the circumferential area of the screw fastened to the receiving section can easily protrude from at least one side in the widthwise direction of the pressing segments.

Furthermore, in the above aspect, the ligament fixation system may further include an installation jig. The screw may be provided with a center hole extending through the screw along a central axis, and a torque applier capable of applying torque around the central axis. The installation jig may include a rod-like first shaft having a longitudinal axis and a tubular second shaft having an inner hole through which the first shaft is extendable in a movable manner along the longitudinal axis and in a rotatable manner around the longitudinal axis. The first shaft may extend through the center hole of the screw, support the screw in a rotatable manner around the longitudinal axis, and have a distal end that is abuttable on an inner surface of the connection segment of the ligament fixation device. The second shaft may include a connector at a distal end thereof. The connector detachably engages with the torque applier of the screw.

According to this configuration, the connector provided at the distal end of the second shaft is engaged with the torque applier provided in the screw, and the inner surface of the connection segment of the ligament fixation device equipped with the reconstruction ligament is brought into abutment with the distal end of the first shaft extending through the inner hole of the second shaft and the center hole of the screw. In this state, the ligament fixation device is inserted into the bone tunnel by being pressed using the first shaft, and is subsequently rotated around the axis of the first shaft while moving the second shaft forward using the first shaft as a guide.

Accordingly, the rotation of the second shaft is applied to the torque applier via the connector, so that the screw is rotated while being moved forward, whereby the screw can be fastened to the receiving section provided at the inner surfaces of the pressing segments. Then, when the reconstruction ligament is completely compressed as a result of the pressing segments being spread away from each other by the screw and the screw is completely fastened to the inner surface of the bone tunnel, the first shaft and the second shaft are removed from the ligament fixation device. Consequently, the ligament fixation device having the reconstruction ligament secured thereto can be retained within the bone tunnel.

Furthermore, in the above aspect, the torque applier may be an engagement section having a noncircular cross-sectional shape provided along the longitudinal axis in at least a part of an inner surface of the center hole. The connector may have a cross-sectional shape complementary to that of the engagement section.

According to this configuration, the torque acting around the longitudinal axis of the bone tunnel can be applied to the screw by simply engaging the torque applier constituted of an engagement section having a noncircular cross section with the connector having a complementary cross-sectional shape.

### REFERENCE SIGNS LIST

1 ligament fixation device
2 connection segment
3 pressing segment
5 receiving section (female threaded section)
7 recess
10 main body
100 ligament fixation system
110 installation jig
121 first shaft
131 inner hole
132 second shaft
133 hexagonal columnar section (connector)
150 screw
151 center hole
152 hexagonal hole (torque applier)
200 reconstruction ligament
A longitudinal axis
B bone
H bone tunnel
L lengthwise direction
S widthwise direction

1. A ligament fixation method comprising:
forming a bone tunnel in a bone;
disposing a reconstruction ligament along an outer surface of a U-shaped ligament fixation device;
inserting the ligament fixation device and the reconstruction ligament into the bone tunnel;
fastening a screw from an opening of the ligament fixation device into the ligament fixation device;
interposing the reconstruction ligament between the ligament fixation device and the bone by spreading opposing ends of the U-shaped ligament fixation device away from each other; and
fixing the reconstruction ligament to the bone screwing a part of the screw into the bone.

2. The ligament fixation method according to claim 1, further comprising:
forming the bone tunnel to a predetermined depth from a surface of the bone;
mounting the reconstruction ligament to the ligament fixation device, the reconstruction ligament being disposed along outer surfaces of a pair of plate-like pressing segments of the ligament fixation device and an outer surface of a connection segment connecting the pair of opposing pressing segments;
inserting the ligament fixation device into the bone tunnel from the connection-segment side; and
fastening the screw to a receiving section of the ligament fixation device to increase a distance between the pair of pressing segments and to compress the reconstruction ligament between each pressing segment and an inner surface of the bone tunnel, and causing the screw protruding in a widthwise direction of the pressing segments to dig into the inner surface of the bone tunnel.

3. The ligament fixation method according to claim 2,
wherein the bone tunnel has a rectangular cross-sectional that is larger than an outer shape of the connection segment.

4. A ligament fixation device for fixing a reconstruction ligament to a bone tunnel, the ligament fixation device comprising:
a main body that includes a pair of plate-like pressing segments and a connection segment connecting the pair of opposing pressing segments; and
a receiving section that is provided in opposing surfaces of the pair of pressing segments such that a screw is configured to fasten onto the receiving section in a lengthwise direction of the pressing segments from a distal side of the pressing segments, wherein:
the receiving section is disposed at a position where the receiving section sets an axis of the screw configured to be fastened at an intermediate location in a widthwise direction of the connection segment, the receiving section is configured to:
cause the pair of pressing segments to deform in a direction that increases a distance between the pressing segments when the screw is fastened to the receiving section, and
allow a circumferential area of the fastened screw to protrude from at least one side in a widthwise direction of the pressing segments, and
the reconstruction ligament is fixed to the bone tunnel by inserting the reconstruction ligament disposed along outer surfaces of the pressing segments and the connection segment into the bone tunnel from the connection-segment side and fastening the screw to the receiving section.

5. The ligament fixation device according to claim 4,
wherein the receiving section is disposed at a position where the receiving section sets the axis of the screw to be fastened at a central location in the widthwise direction of the connection segment and allows the fastened screw to protrude from opposite sides in the widthwise direction of the pressing segments.

6. The ligament fixation device according to claim 4,
wherein the outer surface of each pressing segment is provided with a recess that partially accommodates the reconstruction ligament in the lengthwise direction of the pressing segment.

7. A ligament fixation system comprising:
the ligament fixation device according to claim 4; and
the screw.

8. The ligament fixation system according to claim 7,
wherein the screw has a diameter that is larger than a widthwise dimension of the pair of pressing segments.

9. The ligament fixation system according to claim 7, further comprising:
an installation jig, wherein:
the screw includes a center hole extending through the screw along a central axis, and a torque application section configured to apply torque about the central axis,
the installation jig includes a rod-like first shaft having a longitudinal axis and a tubular second shaft having an inner hole through which the first shaft is configured to extend and rotate along the longitudinal axis,
the first shaft extends through the center hole of the screw, supports the screw in a rotatable manner around the longitudinal axis, and has a distal end that is configured to abut an inner surface of the connection segment of the ligament fixation device, and
the second shaft includes a connector at a distal end, the connector being configured to detachably engage with the torque application portion of the screw.

10. The ligament fixation system according to claim 9, wherein:
the torque application portion is an engagement section having a noncircular cross-sectional shape provided along the longitudinal axis in a part of an inner surface of the center hole, and
the connector has a cross-sectional shape configured to complement the engagement section.
